(19) United States
(12) Plant Patent Application Publication
(10) Pub. No.: US 2011/0239337 P1
(43) Pub. Date: Sep. 29, 2011

(54) Cherry laurel plant named 'Ivory'

(50) ;

(75) Inventor: Fons Custers, Kronenberg (NL)

(73) Assignee: Van Vliet New Plants BV, Stroe (NL)

(21) Appl. No.: 12/798,149

(22) Filed: Mar. 29, 2010

(51) Int. Cl. A01H 5/00

(52) U.S. Cl. PLT/180

(57) ABSTRACT

‘Ivory’ is a new Prunus laurocerasus variety particularly distinguished by having dark green foliage that has cream and greenish white blotches and dots, variegation that consists of small yellow-green and green-white dot, and a relatively narrow, upright growth habit, is disclosed.

### GENUS AND SPECIES

Prunus laurocerasus

### VARIETY DENOMINATION

‘Ivory’

### BACKGROUND OF THE NEW PLANT

The present invention relates to a new and distinct variety of Prunus laurocerasus plant and is hereinafter referred to by the variety name ‘Ivory’. ‘Ivory’ is a naturally-occurring branch mutation of a selection of Prunus laurocerasus ‘Caucasica’ (unpatented) discovered in a cultivated area in Rochester, N.Y. in Summer 2003.

‘Ivory’ has been asexually reproduced for seven generations by softwood cuttings in a controlled environment in Kronenberg, The Netherlands since Summer 2003. ‘Ivory’ has been found to retain its distinctive characteristics through successive generations of asexual propagation via softwood cuttings.

### REFERENCE TO PLANT BREEDERS' RIGHTS

Plant Breeders' Rights for this variety were granted by the European Union on Dec. 15, 2009.

### SUMMARY OF THE INVENTION

The following characteristics of the new variety have been repeatedly observed and can be used to distinguish ‘Ivory’ as a new and distinct variety of Prunus laurocerasus plant:
1. Dark green foliage that has cream and greenish-white blotches and dots;
2. Variegation that consists of small yellow-green and green-white dots; and
3. A relatively narrow, upright growth habit.

### DESCRIPTION OF THE PHOTOGRAPHS

This new Prunus laurocerasus plant is illustrated by the accompanying photographs which show the plant's form and foliage. The colors shown are as true as can be reasonably obtained by conventional photographic procedures. Photographs were taken of a 1-year-old plant in February 2010.

FIG. 1 shows the overall plant habit.

FIG. 2 shows an individual branch.

FIG. 3 shows a close-up of a leaf.

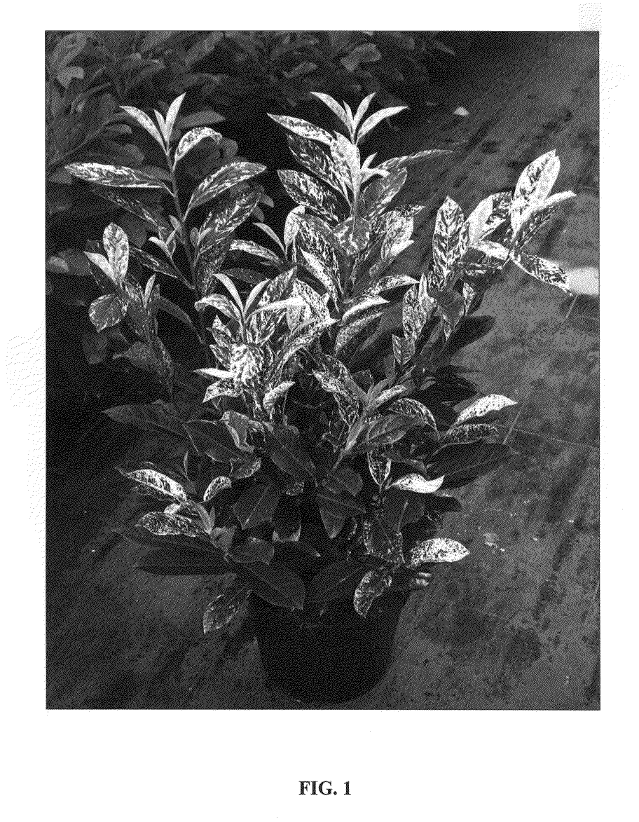

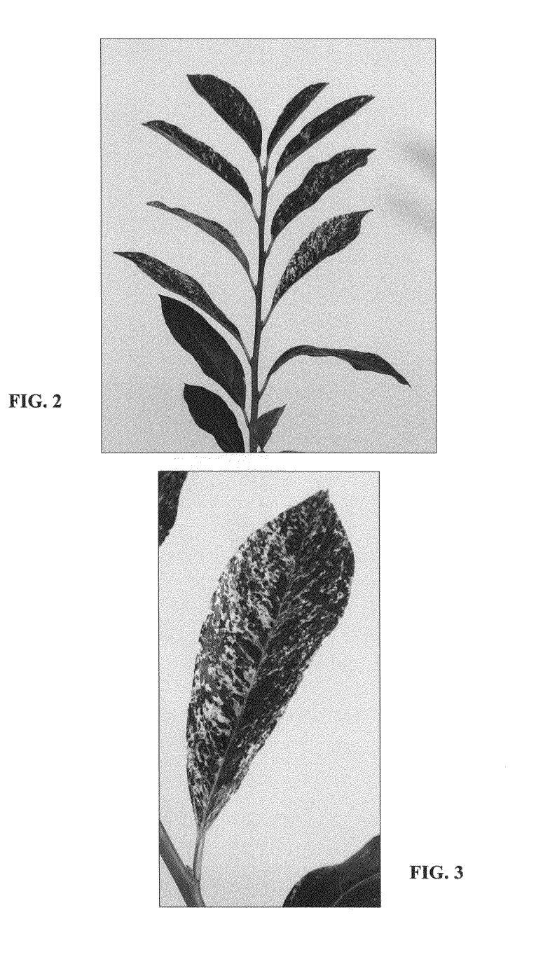

### DESCRIPTION OF THE NEW PLANT

The following detailed description sets forth the distinctive characteristics of ‘Ivory’. The data which define these characteristics were collected from asexual reproductions carried out in Boskoop, The Netherlands in February 2010. The detailed description was taken from 1-year-old plants grown outdoors under normal outdoor light from transplanting of rooted cuttings into 1.5 liter or larger containers. Daytime temperatures ranged from 2° C. to 10° C., while night time temperatures ranged from −5° C. to 1° C. No growth retardants were used.

### DESCRIPTION OF THE NEW PLANT

Classification:
   Botanical name.—Prunus laurocerasus.
   Common name.—Cherry Laurel, English Laurel.
   Denomination.—‘Ivory’.
Parentage:
   Parent.—Naturally-occurring branch mutation of Prunus laurocerasus ‘Caucasica’ (unpatented).
Plant:
   Type.—Perennial, evergreen shrub.
   Propagation type.—Softwood.
   Shape.—Narrow, inverted triangle.
   Growth habit.—Upright.
   Growth rate.—Approximately 25.0 cm per season as a rooted cutting. Once field grown, it has an approximate growth rate of 50.0 cm per season.
   Time to initiate roots.—In summer, approximately 45 days at an average air temperature of 28° C.
   Time to produce a finished plant.—Approximately 3 years from rooted cuttings.
   Roots.—Dense, freely branching, thick, not fleshy, greyed-orange color.
   Size.—Height: 42.2 cm Width (spread): 23.8 cm.
   Vigor.—Moderate to high.
Branches:
   Branching habit.—Moderate, basal branching, pinching not required but will improve branching, only one main stem.
   Appearance of stem.—Rounded, slightly glossy.
   Color.—Young stem: From RHS 143A (Green) to RHS 144A (Yellow-green) Mature stem: RHS 197A (Greyed-green).
   Diameter (average).—0.5 cm.
   Length (average).—38.5 cm.
   Length of internode (average).—2.7 cm.
   Strength.—Very strong.
   Pubescence.—Absent.
Leaves:
   Arrangement.—Alternate, single.
   Fragrance.—Absent.
   Shape.—Obovate to elliptic.
   Apex.—Apiculate.
   Base.—Attenuate.
   Margin.—Slightly serrate (approximately 1 tooth per cm), margins of leaves towards the top of the branch are less serrate and irregularly sinuate.
   Appearance.—Smooth, glossy.
   Mature leaf length (average).—12.6 cm.
   Mature leaf width (average).—4.8 cm.
   Quantity per branch (average).—17.
   Pubescence.—Absent.
   Durability to stresses.—High.
   Color.—Young foliage (upper surface): Darker than RHS 147A (Yellow-green), irregularly blotched and dotted RHS 148B (Yellow-green) and RHS 148D (Yellow-green), RHS 157A (Green-white) and RHS 157D (Green-white), and RHS 192A (Greyed-green) and RHS 192D (Greyed-green) Young foliage (lower surface): RHS 137C (Green), irregularly blotched RHS 192B (Greyed-green) and RHS 192D (Greyed-green) Mature foliage (upper surface): Between RHS 139A (Green) and darker than RHS 147A (Yellow-green), irregularly blotched and dotted RHS 150D (Yellow-green) and RHS 157A (Green-white) Mature foliage (lower surface): RHS 137C (Green) and RHS 137D (Green), irregularly blotched and dotted RHS 157A (Green-white) and RHS 157B (Green-white) to RHS 192A (Greyed-green) and RHS 192C (Greyed-green)
   Variegation.—Present; the lower leaves of a plant are less variegated than those in the middle and the tops of the branches.
   Venation.—Pattern: Pinnate Color: Upper surface: RHS 144B (Yellow-green) Lower surface: RHS 144B (Yellow-green) and RHS 144C (Yellow-green).
   Petiole.—Length (average): 1.5 cm Diameter (average): 0.3 cm Color: Upper surface: RHS 145C (Yellow-green) Lower surface: Darker than RHS 144B (Yellow-green) and RHS 144C (Yellow-green).
Inflorescence: No flowers observed to date.
Fruit and seed set: No seed set observed.
Disease and insect resistance: Not more susceptible to diseases and pests than other Prunus laurocerasus varieties.
Weather tolerance: Plants of the new Prunus laurocerasus have been observed to tolerate high temperatures to at least 35° C. and low temperatures to USDA Zone 6.
Comparison with parental variety: ‘Ivory’ is a new and distinct variety of Prunus laurocerasus having dark green foliage that is blotched and dotted cream color and greenish white color, variegation that consists of small dots, and a relatively narrow, upright growth habit. ‘Ivory’ differs from its parent Prunus laurocerasus ‘Caucasica’ in that ‘Caucasica’ does not have variegated foliage.
Comparison with commercial variety: ‘Ivory’ differs from commercial comparison variety ‘Marbled White’ (not patented) in that ‘Ivory’ has dark green foliage with cream colored and greenish-white colored blotches and dots, variegation that consists of small dots, and a relatively narrow, upright growth habit, while ‘Marbled White’ has dark green leaves with cream colored blotches, variegation that consists of dots and irregular stripes, and a broader, upright growth habit.

I claim:

1. A new and unique variety of Prunus laurocerasus plant named ‘Ivory’ as herein shown and described.
